(19) United States
(12) Patent Application Publication
(10) Pub. No.: US 2024/0366423 A1
(43) Pub. Date: Nov. 7, 2024

(54) AI THERMAL AND HUMAN BODY DETECTION SYSTEM USING TEMPERATURE SENSOR

(71) Applicants: SARANGBANG Co., Ltd, Namyangju-si (KR); MACONE Co.,Ltd, Incheon-si (KR)

(72) Inventors: Hyun Tae KIM, Seongnam-si (KR); Kab Sik SEO, Incheon-si (KR)

(73) Assignees: SARANGBANG Co., Ltd, Namyangju-si (KR); MACONE Co.,Ltd, Incheon-si (KR)

(21) Appl. No.: 18/502,656

(22) Filed: Nov. 6, 2023

(30) Foreign Application Priority Data
May 4, 2023 (KR) 10-2023-0058671

(51) Int. Cl. A61F 7/08

(52) U.S. Cl. CPC A61F 7/08; A61F 2007/0073; A61F 2007/0093; A61F 2007/0096

(57) ABSTRACT

An AI thermal and human body detection system using a temperature sensor includes a base unit; a bimetal cutting off power to prevent overheating; the temperature sensor detecting a temperature of a thermal mat; a heating wire disposed in a zigzag shape on the base unit and converting electrical energy into heat energy; and a power supply unit supplying power to each component of the thermal mat.

### CROSS-REFERENCE TO RELATED APPLICATIONS

This application claims benefit of priority to Korean Patent Application No. 10-2023-0058671 filed May 4, 2023, the entire contents of which are incorporated herein by reference.

### BACKGROUND

### 1. Field

The present invention relates to an AI thermal and a human body detection system using a temperature sensor, and more particularly, to a system that regulates a temperature by using a heating wire and efficiently maintains the temperature by detecting a movement of a human body.

### 2. Description of Related Art

Thermal mats have been mainly used to provide warmth to users by converting electrical energy into heat energy. However, these thermal mats do not detect movements of human bodies and continuously radiated heat even when users are not moving, which causes inefficient energy consumption. In addition, even when users do not move for a long time, thermal mats continue to operate, which causes overheating or discomfort to users.

In addition, there are cases where thermal mats are used for a long period of time after being turned on once or when users go out while thermal mats are turned on but not used. In such cases, thermal mats may overheat and cause a fire.

Various methods have been proposed to solve these problems, but most methods have the disadvantage of having a complicated structure or incurring additional costs. Therefore, there is a need for an efficient thermal mat system having a function of detecting a movement of a user and appropriately adjusting the temperature.

In addition, the thermal products of the related art use various components for temperature control and human body detection, which result in increased complexity and increased costs during a production process, and using a separate component such as a detection plate or a detection wire causes an increase in the defect rate of products and inefficiency in the power consumption and space utilization.

### SUMMARY

The present invention provides a thermal mat system that performs efficient temperature control and reduces energy consumption through temperature detection and human body detection. In addition, the present invention prevents overheating and increases user convenience by detecting a movement of a user.

In addition, the present invention integrates a human body detection function by using a temperature sensor, which is an indispensable component in a thermal product, which does not require a separate detection plate or detection line, which is an additional component for human body detection, thereby reducing the complexity of a production process and reducing costs.

The present invention introduces an AI thermal and human body detection system using a temperature sensor, which greatly improves productivity, thereby reducing the cost for a company, and which reduces a defect rate through simplification of a thermal product, thereby maximizing the competitiveness of the company, and accordingly, enhances a position of the company in a thermal product market and provides better products to consumers.

In an aspect of the present invention, an AI thermal and human body detection system using a temperature sensor includes a base unit; a bimetal cutting off power to prevent overheating; the temperature sensor detecting a temperature of a thermal mat; a heating wire disposed in a zigzag shape on the base unit and converting electrical energy into heat energy; and a power supply unit supplying power to each component of the thermal mat.

The temperature sensor may further include a human body detection unit configured to detect a movement of a human body at one end of the base unit where the heating wire is disposed, the AI thermal and human body detection system may further include a manipulation unit, wherein the manipulation unit includes a user input unit; a storage unit; and a control unit, wherein the control unit, when receiving an operation command at a preset temperature from the user input unit, controls power applied from the power supply unit to be transferred to the heating wire so that the heating wire generates heat energy, controls the temperature sensor to measure a temperature value in an operating period of a preset first cycle formed to alternately repeat a resting period and an operating period among a plurality of cycles, and to detect the movement of the human body in the resting period of the first cycle based on the numerical value measured by the human body detection unit, when the movement of the human body is detected, controls the power supply unit to apply power to the heating wire at a second cycle formed to alternately repeat a preset resting period and an operating period among a plurality of cycles in the operating period of the second cycle, when the movement of the human body is not detected, controls the power supply unit to apply power to the heating wire at a third cycle having a resting period longer than the resting period of the second cycle, and when a time at which the movement of the human body is not detected exceeds a preset time, controls the power supply unit not to apply power to the heating wire.

The human body detection unit may include a potential detection unit detecting a potential signal of power supplied from the temperature sensor, and the potential detection unit may detect the movement of the human body based on determination criteria of a first potential measurement value when the movement of the human body previously stored in the storage unit is not detected and a second potential measurement value when the movement of the human body is detected.

### BRIEF DESCRIPTION OF DRAWINGS

FIG. 1 is a diagram showing a general configuration of an AI thermal and human body detection system using a temperature sensor according to an embodiment of the present invention.

FIG. 2 is a diagram showing a specific configuration of the AI thermal and human body detection system using the temperature sensor according to an embodiment of the present invention.

FIG. 3 is a diagram specifically showing configurations of a manipulation unit of the AI thermal and human body detection system using the temperature sensor according to an embodiment of the present invention.

FIG. 4 is a graph showing a control unit adjusting an operation in the AI thermal and human body detection system using the temperature sensor according to an embodiment of the present invention.

FIG. 5 is a diagram showing a potential detection unit determining a potential measurement value in the AI thermal and human body detection system using the temperature sensor according to an embodiment of the present invention.

### DETAILED DESCRIPTION

Hereinafter, embodiments of the present invention will be described in detail with reference to the accompanying drawings. In the drawings, the same reference numbers or signs refer to components that perform substantially the same function, and the size of each component in the drawings may be exaggerated for clarity and convenience. However, the technical idea and the core configuration and operation of the present invention are not limited only to the configuration or operation described in the following examples. In describing the present invention, when a detailed description 4 well-known technology relating to the present invention may unnecessarily make the spirit of the present invention unclear, a detailed description thereof will be omitted.

In embodiments of the present invention, terms including ordinal numbers such as first and second are used only for the purpose of distinguishing one component from other components, and singular expressions include plural expressions unless the context clearly indicates otherwise. Also, in embodiments of the present invention, it should be understood that terms such as ‘configured’, ‘include’, and ‘have’ do not preclude the existence or addition possibility of one or more other features or numbers, steps, operations, components, parts, or combinations thereof. In addition, in the embodiment of the invention, a ‘module’ or a ‘unit’ performs at least one function or operation, and may be implemented in hardware or software, or a combination of hardware and software, and may be integrated into at least one module and implemented as at least one processor. In addition, in embodiments of the invention, at least one of the plurality of elements refers to not only all of the plurality of elements, but also each one or all combinations thereof excluding the rest of the plurality of elements. An expression “configured (or set) to” used in the present invention may be replaced by an expression “suitable for,” “having the capacity to” “designed to,” “adapted to,” “made to,” or “capable of” depending on a situation. The term “˜configured (or set) to” may not necessarily mean “specifically designed to” in hardware. Instead, an expression “˜an apparatus configured to” may mean that the apparatus “is capable of” together with other apparatuses or components. For example, a “processor configured (or set) to perform A, B, and C” may mean a dedicated processor (for example, an embedded processor) for performing the corresponding operations or a generic-purpose processor (for example, a central processing unit (CPU) or an application processor) that may perform the corresponding operations by executing one or more software programs stored in a memory apparatus.

Hereinafter, embodiments of the present invention will be described in detail with reference to the accompanying drawings. This is to explain in detail to the extent that those skilled in the art can easily practice the invention, and thus, it is to be noted that the technical spirit and scope of the present invention are not limited.

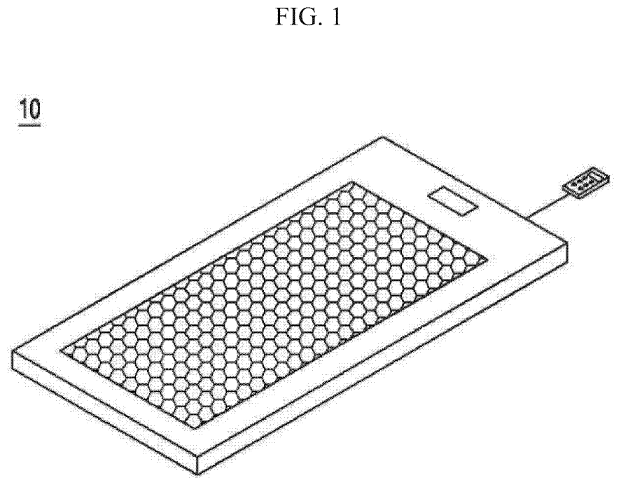

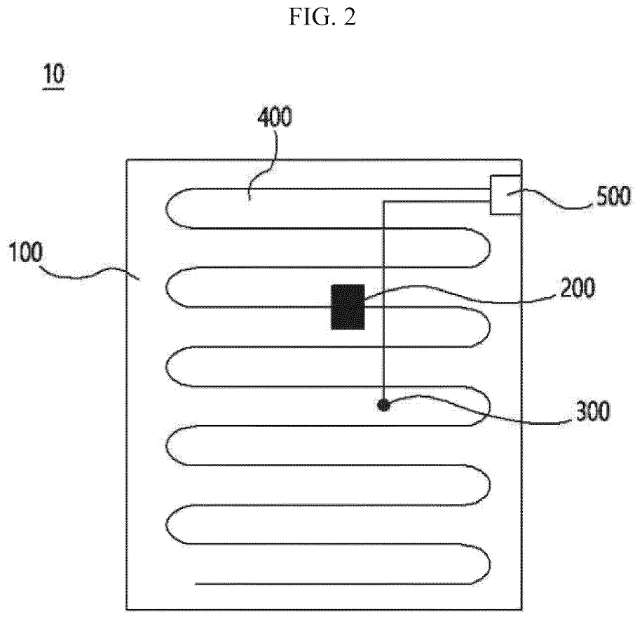

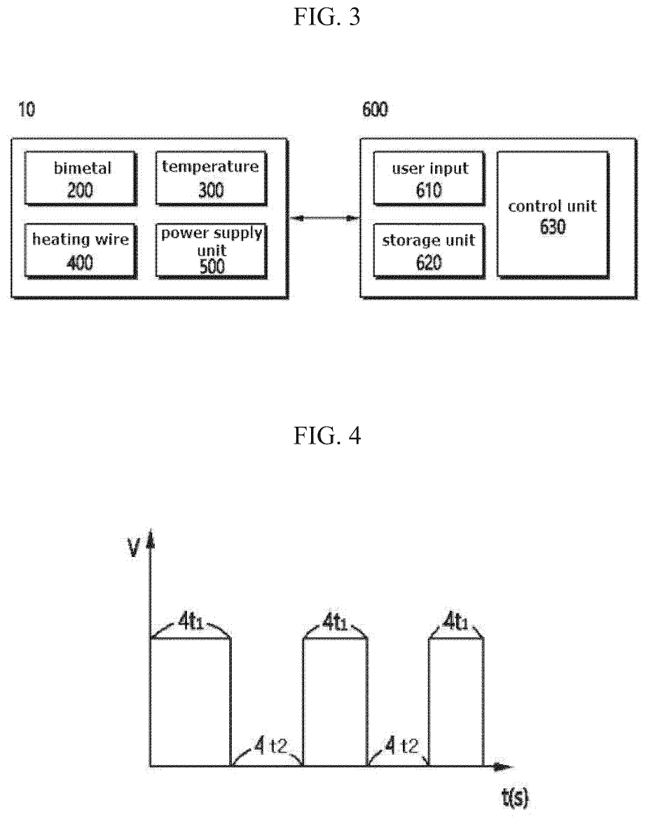

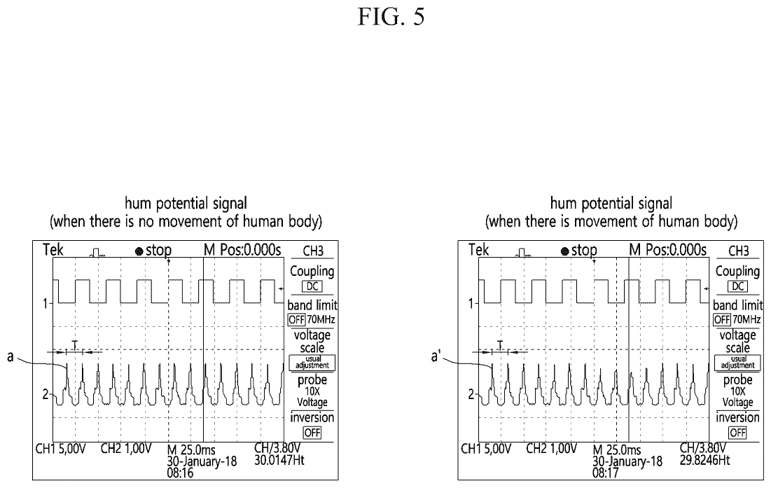

FIG. 1 is a diagram showing a general configuration of an AI thermal and human body detection system using a temperature sensor according to an embodiment of the present invention. FIG. 2 is a diagram showing a specific configuration of the AI thermal and human body detection system using the temperature sensor according to an embodiment of the present invention. FIG. 3 is a diagram specifically showing configurations of a manipulation unit of the AI thermal and human body detection system using the temperature sensor according to an embodiment of the present invention. FIG. 4 is a graph showing a control unit adjusting an operation in the AI thermal and human body detection system using the temperature sensor according to an embodiment of the present invention. FIG. 5 is a diagram showing a potential detection unit determining a potential measurement value in the AI thermal and human body detection system using the temperature sensor according to an embodiment of the present invention.

Referring to FIGS. 1 to 5, the AI thermal and human body detection system using the temperature sensor according to an embodiment of the present invention includes a base unit 100, a bimetal 200, a temperature sensor 300, a heating wire 400, and a power supply unit 500.

A thermal mat 10 according to an embodiment of the present invention refers to a mat-type device that generates heat by using electricity to supply warmth to a user, is generally used under a bed, floor, etc., and refers to a device to maintain health and increase energy efficiency, specially, in winter or areas with cold weather.

On the other hand, the thermal mat 10 according to the present invention may include all thermal mats such as an electric thermal mat having a built-in heating wire 400 made of an electrical conductor and generating heat when electricity passes through the heating wire 400, a carbon thermal mat generating heat by evenly distributing carbon fibers having a high electrical resistance and generating heat when a current flows throughout the mat, a far-infrared thermal mat generating heat by using a far-infrared ray generating device, a self-thermal mat generating heat by using a magnetic field, and a hot pad thermal mat into which a heating agent is inserted to generate heat through a chemical reaction. In other words, the thermal mat 10 according to an embodiment of the present invention may include all various types of thermal mats 10 that may be selected in consideration of electricity usage, heating efficiency, mobility, etc. according to the need and environment of the user and may include any means dissipating heat to the user.

The base unit 100 of the AI thermal and human body detection system using the temperature sensor according to an embodiment of the present invention is a portion that provides a basic structure of the thermal mat 10, and serves as a basic frame in which various configurations included in the thermal mat 10, such as the heating wire 400, the bimetal 200, the temperature sensor 300, etc., are disposed. Meanwhile, it may be desirable for the base unit 100 to be manufactured of a material that is resistant to a high temperature and has a high electrical insulation to improve the durability and stability of the thermal mat 10.

The bimetal 200 of the AI thermal and human body detection system using the temperature sensor according to an embodiment of the present invention serves to cut off power to prevent overheating. More specifically, the bimetal 200 is a material in which two or more metals are joined and may perform functions such as temperature detection and control and overheating prevention based on the characteristics that the bimetal 200 moves or is deformed according to a temperature change by using different thermal expansion coefficients of the respective metals. The bimetal 200 according to the present invention may have various embodiments such as a temperature compensation bimetal, a temperature display bimetal, a snap action bimetal, a bimetal strip, a bimetal coil, etc. However, for convenience of explanation, the present invention is described on the premise of an overheating prevention bimetal that is configured to block an electric circuit by being deformed according to the thermal expansion of the metal when the temperature rises above a certain level.

The temperature sensor 300 of the AI thermal and human body detection system using the temperature sensor according to an embodiment of the present invention is a device that measures temperature by converting physical and chemical changes into electrical signals, and serves to detect the temperature in the thermal mat 10 and detects and measures the temperature to maintain a constant temperature based on the detected temperature. Through this, the constant temperature may be properly maintained in the thermal mat 10.

On the other hand, the temperature sensor 300 according to the present invention may include all of a thermistor that is made of a semiconductor material and has a resistance value changing according to temperature, a thermocouple that measures temperature by using a thermoelectric effect that occurs at a contact point of two different metals, a non-contact infrared (IR) temperature sensor that measures infrared radiant energy emitted from an object, a digital temperature sensor that measures temperature by converting an analog signal into a digital signal, and a fiber optic temperature sensor that measures temperature by using an optical fiber, but for convenience of explanation, the temperature sensor 300 of the present invention is described on the premise of a resistance temperature detector (RTD) method of measuring temperature by using mainly a metal such as platinum, nickel, copper, etc. based on an electrical resistance of metal changing according to the temperature. However, the type of the temperature sensor 300 of the present invention is not limited thereto, and may include all various temperature sensors appropriately selected according to considerations such as a measurement range, precision, reaction speed, environmental conditions, etc.

As the temperature sensor 300 that detects the temperature of the thermal mat 10, a negative temperature coefficient (NTC) sensor, a resistance temperature change (RTD) sensor, a temperature switch, etc. are used. These sensors transmit temperature information by detecting electrical changes due to a temperature change, and a control circuit adjusts the temperature based on the temperature information.

The heating wire 400 of the AI thermal and human body detection system using the temperature sensor according to an embodiment of the present invention is disposed in a zigzag shape on the base unit 100 and serves to convert electrical energy into thermal energy. More specifically, the heating wire 400 refers to a line made of an electrically conductive material that generates heat by using the electrical resistance effect.

Meanwhile, the heating wire 400 according to the present invention may include a nichrome wire which is a high-temperature resistant alloy wire made of nickel (Ni) and chromium (Cr), has excellent corrosion resistance, acid resistance, and heat resistance, and has high electrical resistance, a copper wire which is used in various fields owing to its excellent electrical conductivity and relatively low price, an aluminum wire which is lightweight and has excellent electrical and thermal conductivity, a silicone rubber heating wire which is the flexible heating wire 400 wrapped around a conductive fiber with silicone rubber, has excellent chemical resistance and heat resistance, and may be bent, a carbon fiber heating wire which is a heating wire made of an electrically conductive material using carbon fiber, and has high electrical resistance and excellent thermal conductivity, an induction heating wire which uses a method of generating an electromagnetic field through an electric current and increasing the temperature with heat energy generated therefrom, and a polyvinyl chloride (PVC) heating wire made by insulating the outside thereof with PVC and placing a conductive material inside.

The power supply unit 500 of the AI thermal and human body detection system using the temperature sensor according to an embodiment of the present invention serves to supply power to the components of the thermal mat 10 and regulate voltage and current. For example, the power supply unit 500 according to the present invention may include a voltage converter, a power cord, a plug, etc., and may include auxiliary components having functions such as overvoltage, overcurrent, and short circuit protection through safe power supply.

Meanwhile, the power supply unit 500 according to the present invention may provide a fixed voltage or variable voltage by converting an alternating current voltage generally provided from a household electric socket into a direct current voltage suitable for the thermal mat 10, and may include an AC adapter that adjusts the heating performance of the thermal mat 10 by allowing the user to select a voltage as an additional component.

The temperature sensor 300 of the AI thermal and human body detection system using the temperature sensor according to an embodiment of the present invention further includes a human body detection unit that detects a movement of a human body at one end of the base unit 100 in which the heating wire 400 is disposed.

More specifically, the human body detection unit according to the present invention may use a capacitance check method of determining the presence and movement of the human body by detecting a change in the capacitance that occurs as it gets closer to the human body. In other words, in a capacitance sensor, when the human body approaches electrodes, the capacitance between the electrodes increases, and when the human body moves away from the electrodes, the capacitance decreases, and the movement of the human body is determined by converting such a change into a voltage change.

When such a method is used, the human body detection unit may include an electrode including a pattern made of metal or conductive ink to detect the change in the capacitance, the capacitance sensor including an insulator to block the transfer of electrical energy between electrodes, and a transfer wire that is disposed together with a wire of the temperature sensor 300 and transfers the voltage change according to the change in the capacitance to a control unit 630. Through these, the control unit 630, which will be described below, may interpret the voltage change received from the capacitance sensor and determine whether there is the movement of the human body.

Meanwhile, the human body detection unit may use an electrical capacitive sensor that measures a change in the capacitance by detecting a change in the distance between electrodes according to a pressure change. The electrical capacitive sensor detects pressure according to a deformation of a displacement body between electrodes. For example, when the pressure increases, the displacement body is deformed and the distance between the electrodes decreases so that electrical capacitance increases, and when the pressure decreases, the distance between the electrodes increases and the electrical capacitance decreases. Through this, the control unit 630 interprets the voltage change received from the electrical capacitive sensor and determines whether there is the movement of the human body.

In another embodiment of the present invention, the human body detection unit measures a hum potential having a frequency component of the AC power as an potential that appears on the wire of the temperature sensor 300 by being electrically or magnetically induced by the action of an electric field caused by a commercial AC power applied to the temperature sensor 300, and the control unit 630 may interpret a change in the hum potential and determine whether there is the movement of the human body. FIG. 5 shows such an example, where a denotes a potential signal when there is no movement of the human body, and a′ denotes a potential signal when there is the movement of the human body. However, since such a difference is very small, the change may be interpreted by using an impedance device configured as a resistance device having a high resistance value. Such a difference in the potential signal may be determined based on a database accumulated by repeatedly measuring a result when there is no existing movement of the human body, and measuring a result when there is the movement of the human body.

Meanwhile, the AI thermal and human body detection system using the temperature sensor according to an embodiment of the present invention may be controlled to use artificial intelligence technology to perform feedback on a malfunction in detecting the movement of the human body and to efficiently detect the movement of the human body based on the temperature sensor.

For example, an artificial intelligence (AI) model may perform a data collection step of collecting data of changes in the potential according to the movement of the human body by using a temperature sensor in order for the AI model to recognize and predict patterns in a learning process, a data preprocessing step of refining the collected data, removing noise, and then, extracting important information from the data through feature extraction, and converting the extracted important information into a form suitable for the artificial intelligence model, a model learning step of learning a model through supervised learning or unsupervised learning so as to find the optimal pattern by learning the relationship between the movement of the human body and changes in the potential by using the preprocessed data, a model evaluation step of deriving various evaluation indicators such as accuracy, precision, and recall by using pre-written test data to evaluate the performance of the artificial intelligence model, an algorithm development step of developing algorithm that analyzes changes in the potential in real time and detects the movement of the human body so as to optimize the accurate detection of the movement of the human body, and a system integration and optimization step of integrating the developed movement detection algorithm with the existing temperature sensor system to optimize performance, thereby enhancing and efficiently operating the system that detects the movement of the human body based on the temperature sensor.

More specifically, when describing the application of a convolutional neural network (CNN) to detecting the movement of the human body by using the data measured by the temperature sensor, the CNN may detect the movement of the human body by collecting the data of changes in the potential over time according to the movement of the human body including potential values measured by the temperature sensor, refining the collected data, removing noise, and then, preprocessing the data to be converted into a two-dimensional (2D) image of time-potential using an input of a CNN model, for example, the time as the x-axis and the potential value as the y-axis by converting time series data into the form of the 2D image, constructing the CNN model including a convolution layer that extracts a pattern of the potential value, a pooling layer that increases computational efficiency by reducing the dimension of the data, an activation function, and a fully connected layer, and reconstructing the CNN model by learning and evaluating the CNN model and developing algorithm by using the method described above and integrating the algorithm with the system of the temperature sensor to optimize performance. Meanwhile, using such a CNN model has the effect of making it more intuitive to convert the time series data into an image form and analyze temperature sensor data.

The AI thermal and human body detection system using the temperature sensor according to an embodiment of the present invention includes a manipulation unit 600. The manipulation unit 600 according to the present invention serves to control the entire system operation through the power supply unit 500 and the control unit 630 by receiving input of the user, through which the user may adjust the temperature, operating time, operating mode, etc. of the thermal mat 10.

The manipulation unit 600 according to the present invention includes a user input unit 610, a storage unit 620, and the control unit 630.

The user input unit 610 according to an embodiment of the present invention may include, for example, a keyboard, a mouse, a pen, a voice input device, a touch input device, an infrared camera, a video input device, or any other input device. In addition, besides those for input, the user input unit 610 may include an output device that allows checking input items. The output device may include, for example, one or more of displays, speakers, printers, or other output devices. Additionally, an external input device or output device may be used as the user input unit 610 or an output apparatus. For example, through the user input unit 610, values and modes for various operations of the thermal mat 10 may be input, and whether values and modes are operating properly may be checked through the output device.

The storage unit 620 according to an embodiment of the present invention may store information received through an external computing device connected by wired or wirelessly, receive and store information about a measured temperature value received by the control unit 630 and a numerical value measured by the human body detection unit, which will be described below. The storage unit 620 may store various data according to the processing and control of the control unit 630, which will be described below. The storage unit 620 may be accessed by the control unit 630 to read, record, modify, delete, and update the data. The storage unit 620 may include a non-volatile device such as flash memory, hard-disc drive, and solid-state drive (SSD) so as to retain data regardless of whether the thermal mat system supplies power. In addition, the storage unit 620 may include a volatile memory such as a buffer or RAM temporarily loading data processed by the control unit 630.

The control unit 630 according to an embodiment of the present invention may perform control to operate various components of the thermal mat 10. The control unit 630 may include a control program (or instruction) that allows performing such control operations, an inactive memory in which the control program is installed, a volatile memory in which at least part of the installed control program is loaded, and at least one processor or central processing unit (CPU) executing the loaded control program. In addition, such a control program may be stored in other external electronic devices in addition to the thermal mat 10 and used in connection with the thermal mat 10.

The control program may include program(s) implemented in the form of at least one of BIOS, device driver, operating system, firmware, platform, and application program (application). As an embodiment, the application program may be pre-installed or stored in the storage unit 620 when manufacturing the thermal mat 10, or may receive data of the application program from the outside when used in the future and be stored based on the received data. Meanwhile, the control unit 630 may be implemented in the form of a device, S/W module, circuit, chip, etc., or a combination thereof.

When the control unit 630 according to an embodiment of the present invention receives an operation command at a preset temperature from the user input unit 610, the control unit 630 may control power applied from the power supply unit 500 to be transferred to the heating wire 400 so that the heating wire 400 generates heat energy, and may control the temperature sensor 300 to measure a temperature value in an operating period of a preset first cycle formed to alternately repeat a resting period and the operating period among a plurality of cycles, and to detect the movement of the human body in the resting period of the first cycle based on the numerical value measured by the human body detection unit. That is, the temperature sensor 300 according to the present invention measures the temperature value in the first cycle which is the operating period in which the temperature value is measured in the plurality of cycles in which the resting period and operating period are repeated, and detects the movement of the human body based on a change in the numerical value, that is, the amount of potential, measured through the human body detection unit in the resting period in which the temperature value is not measured. Through this, through the temperature sensor 300, it is possible to simultaneously detect the movement of the human body as well as measure the temperature, thereby reducing manufacturing costs, and improving economic feasibility by simplifying the configuration.

When the movement of the human body is detected, the control unit 630 according to an embodiment of the present invention may control the power supply unit 500 to apply power to the heating wire 400 at a second cycle formed to alternately repeat a preset resting period and an operating period among a plurality of cycles in the operating period of the second cycle, when the movement of the human body is not detected, may control the power supply unit 500 to apply power to the heating wire 400 at a third cycle having a resting period longer than the resting period of the second cycle, and when a time at which the movement of the human body is not detected exceeds a preset time, may control the power supply unit 500 not to apply power to the heating wire 400. In other words, when the movement of the human body is detected, the control unit 630 operates normally, but when the movement of the human body is not detected, it is determined that the user is sleeping well and the resting period is set to be longer to maintain the current temperature. This is to prevent overheating and fire when the temperature is not appropriate, a movement may occur again due to the sleeping user tossing and turning, and at this time, the control unit 630 operates again in a normal cycle at the set temperature, but when the movement is not detected for a long period of time, for example, for more than 4 hours, it is determined that the user went out after sleeping well, and power is no longer applied to the heating wire 400.

The control unit 630 according to an embodiment of the present invention may determine a ratio of the cycle of the heating wire 400 repeating the operating period and the resting period based on various variables such as the set temperature and the area of the thermal mat 10. That is, when the operating period and the resting period are determined at a ratio of 1:k, k may be determined based on [Equation 1] below.

$$k = \frac{{\sum }_{n = 0}^{t} {e}^{1 - n}}{log ( A * T )} \qquad [ Equation 1 ]$$

In [Equation 1] above, k means a ratio constant of the resting period, t means an operating time (minutes) of the thermal mat 10, A means an area (m2) of the thermal mat, and T means a target temperature (° C.) of the thermal mat 10 set by the user. This means that the longer the operating time, the longer the resting period is set, the larger the area, the more likely the heat is to be released, so the shorter the resting period is set, and the higher the target temperature T, the shorter the resting period is set. Meanwhile, such a ratio constant of the resting period may be newly set at intervals of 1 hour or 2 hours according to user settings.

Meanwhile, a movement detection cycle of the temperature sensor 300 also repeats the operating period and resting period. At this time, when the operating period in which temperature is measured and the resting period in which movement is measured are determined at a ratio of 1:k′, k′ may be determined based on [Equation 2] below.

$${k}^{′} = \frac{{\sum }_{m = 0}^{{t}^{′}} {e}^{1 - m}}{log ( A * T )} \qquad [ Equation 2 ]$$

In [Equation 2] above, k′ means a ratio constant of the resting period in which movement of the user is measured, t′ means the number of times the movement is measured at the conventional interval of 1 hour, A means the area (m2) of the thermal mat, and T means the target temperature (° C.) of the thermal mat 10 set by the user. This means that the higher the frequency of movement, the higher the probability that the user is currently positioned on the thermal mat 10, so the greater the number of times the movement is measured in 1 hour, the longer the resting period is set, the larger the area, the more likely the movement is to be measured, so the shorter the rest period is set, and the higher the target temperature T, the higher the probability that the user will sleep well, so the shorter the resting period is set. Meanwhile, such a ratio constant of the resting period may be newly set at intervals of 1 hour or 2 hours according to user settings.

The present invention may provide the thermal mat system that performs efficient temperature control and reduces energy consumption through temperature detection and human body detection, and prevents overheating and increases user convenience by detecting the movement of the user.

In addition, the present invention may integrate the human body detection function by using the temperature sensor, which is the indispensable component in the thermal product, which does not require the separate detection plate or detection line, which is the additional component for human body detection, thereby reducing the complexity of the production process and reducing costs.

The present invention may introduce the AI thermal and human body detection system using the temperature sensor, which greatly improves productivity, thereby reducing the cost for the company, and which reduces the defect rate through simplification of the thermal product, thereby maximizing the competitiveness of the company, and accordingly, enhance the position of the company in the thermal product market and provide better products to consumers.

As described above, the present invention has been described with specific details such as specific components, limited embodiments and drawings, but these are provided only to facilitate a more general understanding of the present invention, the present invention is not limited to the above embodiments, and those skilled in the art may achieve various modifications and variations from the description.

Accordingly, the spirit of the present invention should not be limited to the described embodiments, and the claims described below as well as all modifications equally or equivalent to the claims shall fall within the scope of the spirit of the present invention.

What is claimed is:

1. An AI thermal and human body detection system using a temperature sensor, the AI thermal and human body detection system comprising:
a base unit;
a bimetal cutting off power to prevent overheating;
the temperature sensor detecting a temperature of a thermal mat;
a heating wire disposed in a zigzag shape on the base unit and converting electrical energy into heat energy; and
a power supply unit supplying power to each component of the thermal mat.

2. The AI thermal and human body detection system of claim 1, wherein
the temperature sensor further includes a human body detection unit configured to detect a movement of a human body at one end of the base unit where the heating wire is disposed,
the AI thermal and human body detection system further comprising: a manipulation unit,
wherein the manipulation unit includes
a user input unit; a storage unit; and a control unit,
wherein the control unit,
when receiving an operation command at a preset temperature from the user input unit, controls power applied from the power supply unit to be transferred to the heating wire so that the heating wire generates heat energy,
controls the temperature sensor to measure a temperature value in an operating period of a preset first cycle formed to alternately repeat a resting period and an operating period among a plurality of cycles, and to detect the movement of the human body in the resting period of the first cycle based on the numerical value measured by the human body detection unit,
when the movement of the human body is detected, controls the power supply unit to apply power to the heating wire at a second cycle formed to alternately repeat a preset resting period and an operating period among a plurality of cycles in the operating period of the second cycle, when the movement of the human body is not detected, controls the power supply unit to apply power to the heating wire at a third cycle having a resting period longer than the resting period of the second cycle, and
when a time at which the movement of the human body is not detected exceeds a preset time, controls the power supply unit not to apply power to the heating wire.

3. The AI thermal and human body detection system of claim 2, wherein
the human body detection unit includes a potential detection unit detecting a potential signal of power supplied from the temperature sensor, and
the potential detection unit detects the movement of the human body based on determination criteria of a first potential measurement value when the movement of the human body previously stored in the storage unit is not detected and a second potential measurement value when the movement of the human body is detected.
